(19) United States
(12) Patent Application Publication
(10) Pub. No.: US 2007/0208038 A1
(43) Pub. Date: Sep. 6, 2007

(54) 6-(2-Fluorophenyl)-Triazolopyrimidines, Method for Producing Them, Their use for Controlling Parasitic Fungi and Agents Containing the Same

(75) Inventors: Carsten Blettner, Mannheim (DE); Markus Gewehr, Kastellaun (DE); Wassilios Grammenos, Ludwigshafen (DE); Thomas Grote, Wachenheim (DE); Udo Hunger, Mainz (DE); Bernd Muller, Frankenthal (DE); Matthias Niedenbruck, Limburgerhof (DE); Joachim Rheinheimer, Ludwigshafen (DE); Peter Schafer, Ottersheim (DE); Frank Schieweck, Hessheim (DE); Anja Schwogler, Mannheim (DE); Oliver Wagner, Neustadt (DE); Michael Rack, Heidelberg (DE); Barbara Nave, Deidesheim (DE); Maria Scherer, Godramstein (DE); Siegfried Strathmann, Limburgerhof (DE); Ulrich Schofl, Bruhl (DE); Reinhard Stierl, Freinsheim (DE)

Correspondence Address: BIRCH STEWART KOLASCH & BIRCH, PO BOX 747, FALLS CHURCH, VA, 22040-0747, US

(73) Assignee: BASF Aktiengesellschaft, Ludwigshafen (DE)

(21) Appl. No.: 10/594,738

(22) PCT Filed: Mar. 26, 2005
(86) PCT No.: PCT/EP05/03208

§ 371 Date: Sep. 29, 2006

(30) Foreign Application Priority Data
Mar. 30, 2004 (DE) 10 2004 016 082.1

(51) Int. Cl. A61K 31/519; C07D 487/04; A01N 43/90

(52) U.S. Cl. 514/259.31; 544/262

(57) ABSTRACT

6-(2-Fluorophenyl)-triazolopyrimidines of the formula I 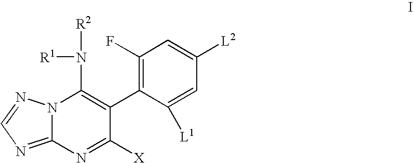 in which the substituents are as defined below:
R¹ is C₄-C₈-alkyl, C₄-C₈-haloalkyl, substituted C₃-C₈-cycloalkyl, C₃-C₈-halocycloalkyl, C₅-C₈-alkenyl, C₂-C₈-haloalkenyl, C₃-C₆-cycloalkenyl, C₃-C₆-halocycloalkenyl, C₂-C₈-alkynyl, C₂-C₈-haloalkynyl or phenyl, naphthyl, or a five- or six-membered saturated, partially unsaturated or aromatic heterocycle which contains one to four heteroatoms from the group consisting of O, N and S,
R² is hydrogen, C₁-C₃-alkyl or one of the groups mentioned under R¹,
   R¹ and R² together with the nitrogen atom to which they are attached may also form a five- or six-membered heterocyclyl or heteroaryl which is attached via N and contain one to three further heteroatoms from the group consisting of O, N and S as ring member, except for piperidin-1-yl optionally substituted by methyl groups;
   R¹ and/or R² may be substituted according to the description;
L¹ is chlorine or fluorine;
L² is hydrogen,
   is, if L¹ is fluorine, also fluorine; X is alkyl; processes for preparing these compounds, compositions comprising them and their use for controlling phytopathogenic harmful fungi.

The present invention relates to 6-(2-fluorophenyl)-triazolopyrimidines of the formula I 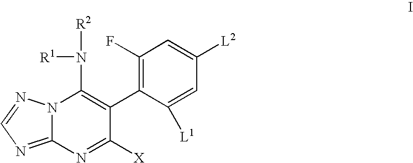
In which the substituents are as defined below:
R¹ is C₄-C₈-alkyl, C₄-C₈-haloalkyl, C₃-C₆-cycloalkyl substituted by at least one group R^{a}, C₃-C₈-halocycloalkyl, C₃-C₆-cycloalkyl-C₁-C₆-alkyl, C₅-C₈-alkenyl, C₂-C₈-haloalkenyl, C₃-C₆-cycloalkenyl, C₃-C₆-halocycloalkenyl, C₂-C₈-alkynyl, C₂-C₈-haloalkynyl or phenyl, naphthyl, or a five- or six-membered saturated, partially unsaturated or aromatic heterocycle which contains one to four heteroatoms from the group consisting of O, N and S,
R² is hydrogen, C₁-C₃-alkyl or one of the groups mentioned under R¹,
   R¹ and R² together with the nitrogen atom to which they are attached may also form a five- to eight-membered saturated or partially unsaturated heterocyclyl or a five- or six-membered heteroaryl which is attached via N and may contain one to three further heteroatoms from the group consisting of O, N and S as ring member and/or may carry one or more substituents from the group consisting of halogen, C₁-C₆-alkyl, C₁-C₆-haloalkyl, C₂-C₆-alkenyl, C₂-C₆-haloalkenyl, C₁-C₆-alkoxy, C₁-C₆-haloalkoxy, C₃-C₆-alkenyloxy, C₃-C₆-haloalkenyloxy, (exo)-C₁-C₆-alkylene and oxy-C₁-C₃-alkyleneoxy,
   except piperidin-1-yl, which is unsubstituted or substituted by one or more methyl groups;
   R¹ and/or R² may carry one to four identical or different groups R^{a}:
   R^{a} is halogen, cyano, nitro, hydroxyl, C₁-C₆-alkyl, C₁-C₆-haloalkyl, C₁-C₆-alkylcarbonyl, C₃-C₆-cycloalkyl, C₁-C₆-alkoxy, C₁-C₆-haloalkoxy, C₁-C₆-alkoxycarbonyl, C₁-C₆-alkylthio, C₁-C₆-alkylamino, di-C₁-C₆-alkylamino, C₂-C₈-alkenyl, C₂-C₈-haloalkenyl, C₂-C₆-alkenyloxy, C₂-C₈-alkynyl, C₂-C₈-haloalkynyl, C₃-C₆-alkynyloxy, oxy-C₁-C₃-alkyleneoxy, C₃-C₈-cycloalkenyl, phenyl, naphthyl, a five- or six-membered saturated, partially unsaturated or aromatic heterocycle which contains one to four heteroatoms from the group consisting of O, N and S, where these aliphatic, alicyclic or aromatic groups for their part may be partially or fully halogenated;
L¹ is chlorine or fluorine;
L² is hydrogen,
   is, if L¹ is fluorine, also fluorine;
X is C₁-C₄-alkyl.

Moreover, the invention relates to a process for preparing these compounds, to compositions comprising them and to their use for controlling phytopathogenic harmful fungi.

5-Alkyl-6-halophenyltriazolopyrimidines are known in a general manner from U.S. Pat. No. 5,994,360. Triazolopyrimidines having optically active amino substituents in the 7-position are proposed in a general manner in WO 02/38565.

The compounds described in the publications mentioned above are suitable for controlling harmful fungi.

However, their action is not always entirely satisfactory in every respect. It is an object of the present invention, therefore, to provide compounds having improved activity and/or a broader activity spectrum.

We have found that this object is achieved by the compounds defined at the outset. Moreover, we have found a process for their preparation, compositions comprising them and methods for controlling harmful fungi using the compounds I.

The compounds according to the invention differ from those described in the abovementioned publication by the specific combination of the substitution in the 5-position and the substitution of the 6-phenyl group with 7-amino groups of the triazolopyrimidine skeleton.

Compared to the known compounds, the compounds of the formula I have increased activity and/or a broader activity spectrum against harmful fungi.

The compounds according to the invention can be obtained by different routes.

Compounds of the formula I can be obtained in an advantageous manner by the following synthesis route:

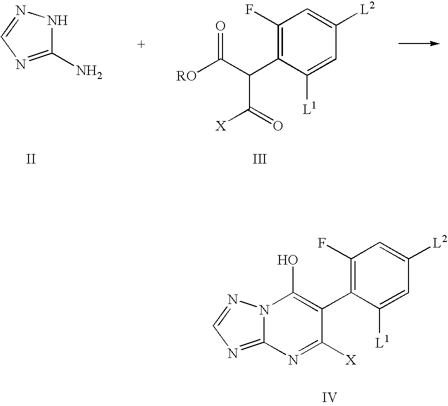

Starting with 5-amino-1,2,4-triazole of the formula II and keto esters III, the 5-alkyl-7-hydroxy-6-phenyltriazolopyrimidines IV are obtained. In the formulae III and IV, X is C₁-C₄-alkyl. Using the easily obtainable 2-phenylacetoacetic esters (III where X¹=CH₃), the 5-methyl-7-hydroxy-6-phenyltriazolopyrimidines are obtained [cf. Chem. Pharm. Bull., 9 (1961), 801]. The preparation of the starting materials III is advantageously carried out under the conditions described in EP-A 10 02 788.

The compounds of the formula IV are novel. Preferred intermediates are
5-methyl-6-(2-chloro-6-fluorophenyl)-[1,2,4]triazolo[1,5-a]pyrimidin-7-ol;
5-methyl-6-(2,6-difluorophenyl)-[1,2,4]triazolo[1,5-a]pyrimidin-7-ol; and
5-methyl-6-(2,4,6-trifluorophenyl)-[1,2,4]triazolo[1,5-a]pyrimidin-7-ol.

The 5-alkyl-7-hydroxy-6-phenyltriazolopyrimidines IV are reacted with halogenating agents [HAL] under the conditions described further above to give the 7-halotriazolopyrimidines of the formula V in which Y is a halogen atom. Preference is given to using chlorinating or brominating agents, such as phosphorus oxybromide, phosphorus oxychloride, thionyl chloride, thionyl bromide or sulfuryl chloride. The reaction can be carried out in the absence or the presence of a solvent. Customary reaction temperatures are from 0 to 150° C. or, preferably, from 80 to 125° C.

The compounds of the formula V are novel. Preferred intermediates are
7-chloro-5-methyl-6-(2-chloro-6-fluorophenyl)-[1,2,4]triazolo[1,5-a]pyrimidine;
7-bromo-5-methyl-6-(2-chloro-6-fluorophenyl)-[1,2,4]triazolo[1,5-a]pyrimidine;
7-chloro-5-methyl-6-(2,6-difluorophenyl)-[1,2,4]triazolo[1,5-a]pyrimidine;
7-bromo-5-methyl-6-(2,6-difluorophenyl)-[1,2,4]triazolo[1,5-a]pyrimidine;
7-chloro-5-methyl-6-(2,4,6-trifluorophenyl)-[1,2,4]triazolo[1,5-a]pyrimidine; and
7-bromo-5-methyl-6-(2,4,6-trifluorophenyl)-[1,2,4]triazolo[1,5-a]pyrimidine.

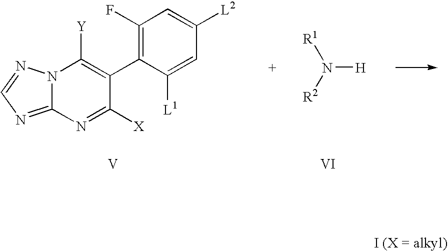

The reaction of V with amines VI is advantageously carried out at from 0° C. to 70° C., preferably from 10° C. to 35° C., preferably in the presence of an inert solvent, such as ethers, for example dioxane, diethyl ether or, in particular, tetrahydrofuran, halogenated hydrocarbons, such as dichloromethane, and aromatic hydrocarbons, such as, for example, toluene [cf. WO-A 98/46608].

Preference is given to using a base, such as tertiary amines, for example triethylamine, or inorganic amines, such as potassium carbonate; it is also possible for excess amine of the formula VI to serve as base.

Alternatively, compounds of the formula I can also be prepared from 5-halotriazolopyrimidines of the formula VII in which X is halogen, in particular chlorine, and malonates of the formula VIII. In the formula VIII, X¹ is hydrogen or C₁-C₃-alkyl and R is C₁-C₄-alkyl. These compounds are converted into compounds of the formula IX and decarboxylated to give compounds I [cf. U.S. Pat. No. 5,994,360].

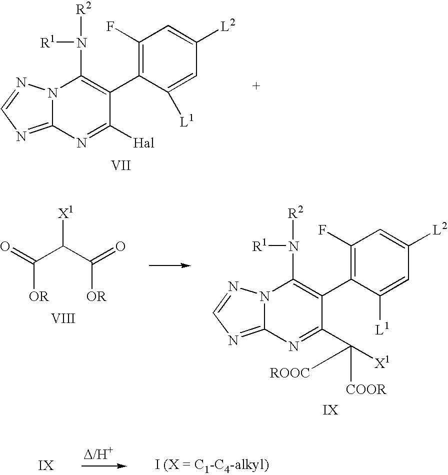

The malonates VIII are known from the literature [J. Am. Chem. Soc. 64 (1942), 2714; J. Org. Chem. 39 (1974), 2172; Helv. Chim. Acta 61 (1978), [565], or they can be prepared in accordance with the literature cited.

The subsequent hydrolysis of the esters IX is carried out under generally customary conditions; depending on the various structural elements, alkaline or acidic hydrolysis of the compounds IX may be advantageous. Under the conditions of ester hydrolysis, there may already be complete or partial decarboxylation to I.

Decarboxylation is usually carried out at temperatures of from 20° C. to 180° C., preferably from 50° C. to 120° C., in an inert solvent, if appropriate in the presence of an acid, which may also serve as solvent.

Suitable acids are hydrochloric acid, sulfuric acid, phosphoric acid, formic acid, acetic acid, p-toluenesulfonic acid. Suitable solvents are water, aliphatic hydrocarbons, such as pentane, hexane, cyclohexane and petroleum ether. Aromatic hydrocarbons, such as toluene, o-, m- and p-xylene, halogenated hydrocarbons, such as methylene chloride, chloroform and chlorobenzene, ethers, such as diethyl ether, diisopropyl ether, tert-butyl methyl ether, dioxane, anisole and tetrahydrofuran, nitriles, such as acetonitrile and propionitrile, ketones, such as acetone, methyl ethyl ketone, diethyl ketone and tert-butyl methyl ketone, alcohols, such as methanol, ethanol, n-propanol, isopropanol, n-butanol and tert-butanol, and also dimethyl sulfoxide, dimethylformamide and dimethylacetamide; with particular preference, the reaction is carried out in hydrochloric acid or acetic acid. It is also possible to use mixtures of the solvents mentioned.

The compounds of the formula VII are known in a general manner from EP-A 550 113 or WO 98/46608 or can be obtained analogously to the methods described therein.

Compounds of the formula I can also be obtained by coupling 5-halotriazolopyrimidines of the formula VII with organometallic reagents of the formula X. In one embodiment of this process, the reaction is carried out with transition metal catalysis, such as Ni or Pd catalysis.
VII+M^{y}(—X²)_{y→I} X

In formula X, M is a metal ion of valency Y, such as, for example, B, Zn or Sn, and X² is C₁-C₃-alkyl. This reaction can be carried out, for example, analogously to the following methods: J. Chem. Soc. Perkin Trans. 1, (1994), 1187, ibid. 1, (1996) 2345; WO-A 99/41255; Aust. J. Chem. 43 (1990), 733; J. Org. Chem. 43 (1978), 358; J. Chem. Soc. Chem. Commun. (1979), 866; Tetrahedron Lett. 34 (1993), 8267; ibid., 33 (1992), 413.

The reaction mixtures are worked up in a customary manner, for example by mixing with water, separating the phases and, if appropriate, chromatographic purification of the crude products. Some of the intermediates and end products are obtained in the form of colorless or slightly brownish viscous oils which are purified or freed from volatile components under reduced pressure and at moderately elevated temperature. If the intermediates and end products are obtained as solids, purification can also be carried out by recrystallization or digestion.

If individual compounds I cannot be obtained by the routes described above, they can be prepared by derivatization of other compounds I.

If the synthesis yields mixtures of isomers, a separation is, however, generally not necessarily required since in some cases the individual isomers can be interconverted during work-up for use or during application (for example under the action of light, acids or bases). Such conversions may also take place after use, for example in the treatment of plants in the treated plant, or in the harmful fungus to be controlled.

In the definitions of the symbols given in the formulae above, collective terms were used which are generally representative of the following substituents:

halogen: fluorine, chlorine, bromine and iodine;

alkyl: saturated straight-chain or branched hydrocarbon radicals having 1 to 4, 6 or 8 carbon atoms, for example C₁-C₆-alkyl such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl;

haloalkyl: straight-chain or branched alkyl groups having 1 to 2, 4, 6 or 8 carbon atoms (as mentioned above), where in these groups some or all of the hydrogen atoms may be replaced by halogen atoms as mentioned above; in particular, C₁-C₂-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl or 1,1,1-trifluoroprop-2-yl;

alkenyl: unsaturated straight-chain or branched hydrocarbon radicals having 2 to 4, 6, 8 or 10 carbon atoms and one or two double bonds in any position, for example C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;

haloalkenyl: unsaturated straight-chain or branched hydrocarbon radicals having 2 to 8 carbon atoms and one or two double bonds in any position (as mentioned above), where in these groups some or all of the hydrogen atoms may be replaced by halogen atoms as mentioned above, in particular by fluorine, chlorine and bromine;

alkynyl: straight-chain or branched hydrocarbon groups having 2 to 4, 6 or 8 carbon atoms and one or two triple bonds in any position, for example C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;

cycloalkyl: mono- or bicyclic saturated hydrocarbon groups having 3 to 6 or 8 carbon ring members, for example C₃-C₈-cycloalkyl such as cyclopropyl, cyclobutyl, cyclopentyl, cyclohexyl, cycloheptyl and cyclooctyl;

five- to six-membered saturated, partially unsaturated or aromatic heterocycle which contains one to four heteroatoms from the group consisting of O, N and S:

5- or 6-membered heterocyclyl which contains one to three nitrogen atoms and/or one oxygen or sulfur atom or one or two oxygen and/or sulfur atoms, for example 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 2-pyrrolin-2-yl, 2-pyrrolin-3-yl, 3-pyrrolin-2-yl, 3-pyrrolin-3-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl and 2-piperazinyl;
5-membered heteroaryl which contains one to four nitrogen atoms or one to three nitrogen atoms and one sulfur or oxygen atom: 5-membered heteroaryl groups which, in addition to carbon atoms, may contain one to four nitrogen atoms or one to three nitrogen atoms and one sulfur or oxygen atom as ring members, for example 2-furyl, 3-furyl, 2-thienyl, 3-thienyl, 1-pyrrolyl, 2-pyrrolyl, 3-pyrrolyl, 1-pyrazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 1-imidazolyl, 2-imidazolyl, 4-imidazolyl and 1,3,4-triazol-2-yl;
6-membered heteroaryl which contains one to three or one to four nitrogen atoms: 6-membered heteroaryl groups which, in addition to carbon atoms, may contain one to three or one to four nitrogen atoms as ring members, for example 2-pyridinyl, 3-pyridinyl, 4-pyridinyl, 3-pyridazinyl, 4-pyridazinyl, 2-pyrimidinyl, 4-pyrimidinyl, 5-pyrimidinyl and 2-pyrazinyl;
   alkylene: saturated straight-chain or branched hydrocarbon radicals having 1 to 4 or 6 carbon atoms, which radicals are attached to the skeleton via a double bond, for example ═CH₂, ═CH—CH₃, ═CH—CH₂—CH₃;
   oxyalkyleneoxy: divalent unbranched chains of 1 to 3 CH₂ groups, where both valencies are attached to the skeleton via an oxygen atom, for example OCH₂O, OCH₂CH₂O and OCH₂CH₂CH₂O.

The scope of the present invention includes the (R)- and (S)-isomers and the racemates of compounds of the formula I having chiral centers.

With a view to the intended use of the triazolopyrimidines of the formula I, particular preference is given to the following meanings of the substituents, in each case on their own or in combination:

Preference is given to compounds I in which R¹ is a group A:
in which 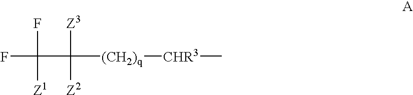
Z¹ is hydrogen, fluorine or C₁-C₆-fluoroalkyl,
Z², Z³ is hydrogen or fluorine, or
Z¹ and Z² together form a double bond;
   q is 1, 2 or 3; and
   R³ is hydrogen or methyl.

In addition, preference is also given to compounds I in which R¹ is C₄-C₈-alkyl or C₄-C₈-haloalkyl.

Preference is furthermore given to compounds I in which R¹ is C₃-C₆-cycloalkyl substituted by at least one group R¹ or is C₃-C₈-halocycloalkyl or C₃-C₆-cycloalkyl-C₁-C₆-alkyl.

Particular preference is given to compounds I in which R¹ is C₃-C₆-cycloalkyl which is substituted by C₁-C₄-alkyl, in particular methyl.

Particular preference is given to compounds I in which R² is hydrogen.

Preference is likewise given to compounds I in which R² is methyl or ethyl.

If R¹ and/or R² comprise haloalkyl or haloalkenyl groups having a center of chirality, the (S)-isomers are preferred for these groups. In the case of halogen-free alkyl or alkenyl groups having a center of chirality in R¹ or R², preference is given to the (R)-configured isomers.

A preferred embodiment of the invention relates to compounds of the formula I.1: 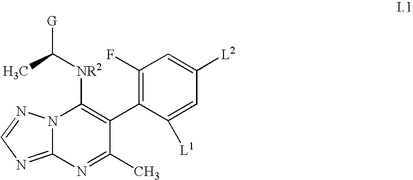
in which
G is C₂-C₆-alkyl, in particular ethyl, n- or isopropyl, n-, sec-, tert-butyl, and C₁-C₄-alkoxymethyl, in particular ethoxymethyl, or C₃-C₆-cycloalkyl, in particular cyclopropyl, cyclopentyl or cyclohexyl; and
R² is hydrogen or methyl.

A further preferred embodiment of the invention relates to compounds of the formula I.2. 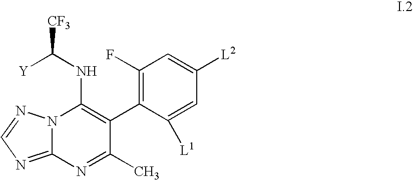
in which Y is C₂-C₄-alkyl, in particular ethyl or propyl.

A further preferred embodiment of the invention relates to compounds in which R¹ and R² together with the nitrogen atom to which they are attached form a five- or six-membered heterocyclyl or heteroaryl which is attached via N and may contain a further heteroatom from the group consisting of O, N and S as ring member and/or may carry one or more substituents from the group consisting of halogen, C₁-C₆-alkyl, C₁-C₆-haloalkyl, C₂-C₆-alkenyl, C₂-C₆-haloalkenyl, C₁-C₆-alkoxy, C₁-C₆-haloalkoxy, C₃-C₆-alkenyloxy, C₃-C₆-haloalkenyloxy, C₁-C₆-alkylene and oxy-C₁-C₃-alkyleneoxy. These compounds correspond in particular to formula I.3, 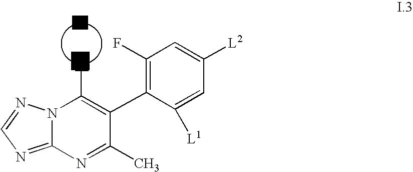
in which
D together with the nitrogen atom forms a five- or six-membered heterocyclyl or heteroaryl which is attached via N and may contain a further heteroatom from the group consisting of O, N and S as ring member and/or may carry one or more substituents from the group consisting of halogen, C₁-C₆-alkyl, C₁-C₆-haloalkyl, C₂-C₆-alkenyl, C₂-C₆-haloalkenyl, C₁-C₆-alkoxy, C₁-C₆-haloalkoxy, C₃-C₆-alkenyloxy, C₃-C₆-haloalkenyloxy, (exo)-C₁-C₆-alkylene and oxy-C₁-C₃-alkyleneoxy.

Particular preference is given to compounds of the formula I, in particular to those of the formula I.3, in which the groups L¹ and L² are as defined below:

L¹ and L² are fluorine,

L¹ is fluorine and L² is hydrogen; or L¹ is chlorine.

Preference is furthermore given to compounds of the formula I.4 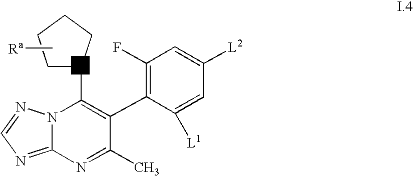
in which the variables are as defined in the formula 1.

Preference is furthermore given to compounds I in which R¹ and R² together with the nitrogen atom to which they are attached form a morpholinyl or thiomorpholinyl ring, in particular a ring which, if appropriate, is substituted by one to three halogen, C₁-C₄-alkyl or C₁-C₄-haloalkyl groups. Particularly preferred are the compounds in which R¹ and R² together with the nitrogen atom to which they are attached form a morpholinyl or a pyrrolidinyl ring, in particular a pyrrolidinyl ring.

The invention furthermore preferably provides compounds I in which R¹ and R² together with the nitrogen atom to which they are attached form a pyrazole ring which, if appropriate, is substituted by one or two halogen, C₁-C₄-alkyl or C₁-C₄-haloalkyl groups, in particular by 3,5-dimethyl or 3,5-di(trifluoromethyl).

In addition, particular preference is also given to compounds of the formula I in which R¹ is CH(CH₃)—CH₂CH₃, CH(CH₃)—CH(CH₃)₂, CH(CH₃)—C(CH₃)₃, CH(CH₃)—CF₃, CH₂C(CH₃)═CH₂, CH₂CH═CH₂; R² is hydrogen or methyl; or R¹ and R² together are —(CH₂)₂CH(CF₃)(CH₂)₂— or —(CH₂)₂—O—(CH₂)₂—.

Particular preference is furthermore given to compounds I in which X is methyl.

In particular with a view to their use, preference is given to the compounds I compiled in the tables below. Moreover, the groups mentioned for a substituent in these tables are per se, independently of the combination in which they are mentioned, a particularly preferred embodiment of the substituent in question.

Table 1

Compounds of the formula I, in which L¹ and L² are fluorine, X is methyl and the combination of R¹ and R² corresponds for each compound to one row of Table A

Table 2

Compounds of the formula I, in which L¹ is chlorine, L² is hydrogen, X is methyl and the combination of R¹ and R² corresponds for each compound to one row of Table A

Table 3

Compounds of the formula I, in which L¹ is fluorine and L² is hydrogen, X is methyl and the combination of R¹ and R² corresponds for each compound to one row of Table A

TABLE A
Compounds of the formula I

| No. | R¹ | R² |
|---|---|---|
| A-1 | CH₂CH₂CH₂CH₃ | H |
| A-2 | CH₂CH₂CH₂CH₃ | CH₃ |
| A-3 | CH₂CH₂CH₂CH₃ | CH₂CH₃ |
| A-4 | CH₂CH₂CH₂CH₃ | CH₂CH₂CH₃ |
| A-5 | CH₂CH₂CH₂CH₃ | CH₂CH₂CH₂CH₃ |
| A-6 | (±) CH(CH₃)—CH₂CH₃ | H |
| A-7 | (±) CH(CH₃)—CH₂CH₃ | CH₃ |
| A-8 | (±) CH(CH₃)—CH₂CH₃ | CH₂CH₃ |
| A-9 | (S) CH(CH₃)—CH₂CH₃ | H |
| A-10 | (S) CH(CH₃)—CH₂CH₃ | CH₃ |
| A-11 | (S) CH(CH₃)—CH₂CH₃ | CH₂CH₃ |
| A-12 | (R) CH(CH₃)—CH₂CH₃ | H |
| A-13 | (R) CH(CH₃)—CH₂CH₃ | CH₃ |
| A-14 | (R) CH(CH₃)—CH₂CH₃ | CH₂CH₃ |
| A-15 | (±) CH(CH₃)—CH(CH₃)₂ | H |
| A-16 | (±) CH(CH₃)—CH(CH₃)₂ | CH₃ |
| A-17 | (±) CH(CH₃)—CH(CH₃)₂ | CH₂CH₃ |
| A-18 | (S) CH(CH₃)—CH(CH₃)₂ | H |
| A-19 | (S) CH(CH₃)—CH(CH₃)₂ | CH₃ |
| A-20 | (S) CH(CH₃)—CH(CH₃)₂ | CH₂CH₃ |
| A-21 | (R) CH(CH₃)—CH(CH₃)₂ | H |
| A-22 | (R) CH(CH₃)—CH(CH₃)₂ | CH₃ |
| A-23 | (R) CH(CH₃)—CH(CH₃)₂ | CH₂CH₃ |
| A-24 | (±) CH(CH₃)—C(CH₃)₃ | H |
| A-25 | (±) CH(CH₃)—C(CH₃)₃ | CH₃ |
| A-26 | (±) CH(CH₃)—C(CH₃)₃ | CH₂CH₃ |
| A-27 | (S) CH(CH₃)—C(CH₃)₃ | H |
| A-28 | (S) CH(CH₃)—C(CH₃)₃ | CH₃ |
| A-29 | (S) CH(CH₃)—C(CH₃)₃ | CH₂CH₃ |
| A-30 | (R) CH(CH₃)—C(CH₃)₃ | H |
| A-31 | (R) CH(CH₃)—C(CH₃)₃ | CH₃ |
| A-32 | (R) CH(CH₃)—C(CH₃)₃ | CH₂CH₃ |
| A-33 | CH₂—C≡CH | H |
| A-34 | CH₂—C≡CH | CH₃ |
| A-35 | CH₂—C≡CH | CH₂CH₃ |

| | |
|---|---|
| A-36 | —(CH₂)₂CH═CHCH₂— |
| A-37 | —(CH₂)₂C(CH₃)═CHCH₂— |
| A-38 | —(CH₂)₃CHFCH₂— |
| A-39 | —(CH₂)₂CHF(CH₂)₂— |
| A-40 | —CH₂CHF(CH₂)₃— |
| A-41 | —(CH₂)₂CH(CF₃)(CH₂)₂— |
| A-42 | —(CH₂)₂O(CH₂)₂— |
| A-43 | —(CH₂)₂S(CH₂)₂— |
| A-44 | —(CH₂)₄— |
| A-45 | —CH₂CH═CHCH₂— |
| A-46 | —CH(CH₃)(CH₂)₃— |
| A-47 | —CH₂CH(CH₃)(CH₂)₂— |
| A-48 | —CH(CH₃)—(CH₂)₂—CH(CH₃)— |
| A-49 | —CH(CH₂CH₃)—(CH₂)₄— |
| A-50 | —(CH₂)₂—CHOH—(CH₂)₂— |
| A-51 | —(CH₂)—CH═CH—(CH₂)₂— |
| A-52 | —(CH₂)₆— |
| A-53 | —CH(CH₃)—(CH₂)₅— |
| A-54 | —(CH₂)₂—N(CH₃)—(CH₂)₂— |
| A-55 | —N═CH—CH═CH— |
| A-56 | —N═C(CH₃)—CH═C(CH₃)— |
| A-57 | —N═C(CF₃)—CH═C(CF₃)— |

The compounds I are suitable as fungicides. They are distinguished by an outstanding effectiveness against a broad spectrum of phytopathogenic fungi, especially from the classes of the Ascomycetes, Deuteromycetes, Oomycetes and Basidiomycetes. Some are systemically effective and they can be used in plant protection as foliar fungicides, as fungicides for seed dressing and as soil fungicides.

They are particularly important in the control of a multitude of fungi on various cultivated plants, such as wheat, rye, barley, oats, rice, maize, grass, bananas, cotton, soya, coffee, sugar cane, vines, fruits and ornamental plants, and vegetables, such as cucumbers, beans, tomatoes, potatoes and cucurbits, and on the seeds of these plants.

They are especially suitable for controlling the following plant diseases:
Alternaria species on fruit and vegetables,
Bipolaris and Drechslera species on cereals, rice and lawns,
Blumeria graminis (powdery mildew) on cereals,
Botrytis cinerea (gray mold) on strawberries, vegetables, ornamental plants and grapevines,
Erysiphe cichoracearum and Sphaerotheca fuliginea on cucurbits,
Fusarium and Verticillium species on various plants,
Mycosphaerella species on cereals, bananas and peanuts,
Phakopsora pachyrhizi und P. meibomiae on soya,
Phytophthora infestans on potatoes and tomatoes,
Plasmopara viticola on grapevines,
Podosphaera leucotricha on apples,
Pseudocercosporella herpotrichoides on wheat and barley,
Pseudoperonospora species on hops and cucumbers,
Puccinia species on cereals,
Pyricularia oryzae on rice,
Rhizoctonia species on cotton, rice and lawns,
Septoria tritici and Stagonospora nodorum on wheat,
Uncinula necatoron grapevines,
Ustilago species on cereals and sugar cane, and
Venturia species (scab) on apples and pears.

The compounds I are also suitable for controlling harmful fungi, such as Paecilomyces variotii, in the protection of materials (e.g. wood, paper, paint dispersions, fibers or fabrics) and in the protection of stored products.

The compounds I are employed by treating the fungi or the plants, seeds, materials or soil to be protected from fungal attack with a fungicidally effective amount of the active compounds. The application can be carried out both before and after the infection of the materials, plants or seeds by the fungi.

The fungicidal compositions generally comprise between 0.1 and 95%, preferably between 0.5 and 90%, by weight of active compound.

When employed in plant protection, the amounts applied are, depending on the kind of effect desired, between 0.01 and 2.0 kg of active compound per ha.

In seed treatment, amounts of active compound of 1 to 1000 g/100 kg, preferably 5 to 100 g, per 100 kilogram of seed are generally required.

When used in the protection of materials or stored products, the amount of active compound applied depends on the kind of application area and on the desired effect. Amounts customarily applied in the protection of materials are, for example, 0.001 g to 2 kg, preferably 0.005 g to 1 kg, of active compound per cubic meter of treated material.

The compounds I can be converted into the customary formulations, for example solutions, emulsions, suspensions, dusts, powders, pastes and granules. The application form depends on the particular purpose; in each case, it should ensure a fine and uniform distribution of the compound according to the invention.

The formulations are prepared in a known manner, for example by extending the active compound with solvents and/or carriers, if desired using emulsifiers and dispersants. Solvents/auxiliaries which are suitable are essentially:
water, aromatic solvents (for example Solvesso products, xylene), paraffins (for example mineral oil fractions), alcohols (for example methanol, butanol, pentanol, benzyl alcohol), ketones (for example cyclohexanone, gamma-butyrolactone), pyrrolidones (NMP, NOP), acetates (glycol diacetate), glycols, fatty acid dimethylamides, fatty acids and fatty acid esters. In principle, solvent mixtures may also be used,
carriers such as ground natural minerals (for example kaolins, clays, talc, chalk) and ground synthetic minerals (for example highly disperse silica, silicates); emulsifiers such as nonionic and anionic emulsifiers (for example polyoxyethylene fatty alcohol ethers, alkylsulfonates and arylsulfonates) and dispersants such as lignosulfite waste liquors and methylcellulose.

Suitable surfactants are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenol ether, ethoxylated isooctylphenol, octylphenol, nonylphenol, alkylphenol polyglycol ethers, tributylphenyl polyglycol ether, tristearylphenyl polyglycol ether, alkylaryl polyether alcohols, alcohol and fatty alcohol/ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, strongly polar solvents, for example dimethyl sulfoxide, N-methylpyrrolidone and water.

Powders, materials for spreading and dustable products can be prepared by mixing or concomitantly grinding the active substances with a solid carrier.

Granules, for example coated granules, impregnated granules and homogeneous granules, can be prepared by binding the active compounds to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active compound. The active compounds are employed in a purity of from 90% to 100%, preferably 95% to 100% (according to NMR spectrum).

The following are examples of formulations: 1. Products for dilution with water

A Water-Soluble Concentrates (SL)

10 parts by weight of a compound according to the invention are dissolved in water or in a water-soluble solvent. As an alternative, wetters or other auxiliaries are added. The active compound dissolves upon dilution with water.

B Dispersible Concentrates (DC)

20 parts by weight of a compound according to the invention are dissolved in cyclohexanone with addition of a dispersant, for example polyvinylpyrrolidone. Dilution with water gives a dispersion.

C Emulsifiable Concentrates (EC)

15 parts by weight of a compound according to the invention are dissolved in xylene with addition of calcium dodecylbenzenesulfonate and castor oil ethoxylate (in each case 5%). Dilution with water gives an emulsion.

D Emulsions (EW, EO)

40 parts by weight of a compound according to the invention are dissolved in xylene with addition of calcium dodecylbenzenesulfonate and castor oil ethoxylate (in each case 5%). This mixture is introduced into water by means of an emulsifying machine (Ultraturrax) and made into a homogeneous emulsion. Dilution with water gives an emulsion.

E Suspensions (SC, OD)

In an agitated ball mill, 20 parts by weight of a compound according to the invention are comminuted with addition of dispersants, wetters and water or an organic solvent to give a fine active compound suspension. Dilution with water gives a stable suspension of the active compound.

F Water-Dispersible Granules and Water-Soluble Granules (WG, SG)

50 parts by weight of a compound according to the invention are ground finely with addition of dispersants and wetters and made into water-dispersible or water-soluble granules by means of technical appliances (for example extrusion, spray tower, fluidized bed). Dilution with water gives a stable dispersion or solution of the active compound.

G Water-Dispersible Powders and Water-Soluble Powders (WP, SP)

75 parts by weight of a compound according to the invention are ground in a rotor-stator mill with addition of dispersants, wetters and silica gel. Dilution with water gives a stable dispersion or solution of the active compound.

2. Products to be Applied Undiluted

H Dustable Powders (DP)

5 parts by weight of a compound according to the invention are ground finely and mixed intimately with 95% of finely divided kaolin. This gives a dustable product.

I Granules (GR, FG, GG, MG)

0.5 part by weight of a compound according to the invention is ground finely and associated with 95.5% carriers. Current methods are extrusion, spray-drying or the fluidized bed. This gives granules to be applied undiluted.

J ULV solutions (UL)

10 parts by weight of a compound according to the invention are dissolved in an organic solvent, for example xylene. This gives a product to be applied undiluted.

The active compounds can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; the intention is to ensure in each case the finest possible distribution of the active compounds according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. Alternatively, it is possible to prepare concentrates composed of active substance, wetter, tackifier, dispersant or emulsifier and, if appropriate, solvent or oil, and such concentrates are suitable for dilution with water.

The active compound concentrations in the ready-to-use preparations can be varied within relatively wide ranges. In general, they are from 0.0001 to 10%, preferably from 0.01 to 1%.

The active compounds may also be used successfully in the ultra-low-volume process (ULV), by which it is possible to apply formulations comprising over 95% by weight of active compound, or even to apply the active compound without additives.

Various types of oils, wetters, adjuvants, herbicides, fungicides, other pesticides, or bactericides may be added to the active compounds, if appropriate not until immediately prior to use (tank mix). These agents can be admixed with the agents according to the invention in a weight ratio of 1:10 to 10:1.

The compositions according to the invention can, in the use form as fungicides, also be present together with other active compounds, e.g. with herbicides, insecticides, growth regulators, fungicides or else with fertilizers. Mixing the compounds I or the compositions comprising them in the application form as fungicides with other fungicides results in many cases in an expansion of the fungicidal spectrum of activity being obtained.

The following list of fungicides, in conjunction with which the compounds according to the invention can be used, is intended to illustrate the possible combinations but does not limit them:
acylalanines, such as benalaxyl, metalaxyl, ofurace or oxadixyl,
amine derivatives, such as aldimorph, dodine, dodemorph, fenpropimorph, fenpropidin, guazatine, iminoctadine, spiroxamine or tridemorph,
anilinopyrimidines, such as pyrimethanil, mepanipyrim or cyprodinyl,
antibiotics, such as cycloheximide, griseofulvin, kasugamycin, natamycin, polyoxin or streptomycin,
azoles, such as bitertanol, bromoconazole, cyproconazole, difenoconazole, dinitroconazole, enilconazole, epoxiconazole, fenbuconazole, fluquiconazole, flusilazole, flutriapole, hexaconazole, imazalil, ipconazole, metconazole, myclobutanil, penconazole, propiconazole, prochloraz, prothioconazole, simeconazole, tebuconazole, tetraconazole, triadimefon, triadimenol, triflumizole or triticonazole,
dicarboximides, such as iprodione, myclozolin, procymidone or vinclozolin,
dithiocarbamates, such as ferbam, nabam, maneb, mancozeb, metam, metiram, propineb, polycarbamate, thiram, ziram or zineb,
heterocyclic compounds, such as anilazine, benomyl, boscalid, carbendazim, carboxin, oxycarboxin, cyazofamid, dazomet, dithianon, famoxadone, fenamidone, fenarimol, fuberidazole, flutolanil, furametpyr, isoprothiolane, mepronil, nuarimol, picobenzamide probenazole, proquinazid, pyrifenox, pyroquilon, quinoxyfen, silthiofam, thiabendazole, thifluzamide, thiophanate-methyl, tiadinil, tricyclazole or triforine,
copper fungicides, such as Bordeaux mixture, copper acetate, copper oxychloride or basic copper sulfate,
nitrophenyl derivatives, such as binapacryl, dinocap, dinobuton or nitrophthal-isopropyl,
phenylpyrroles, such as fenpiclonil or fludioxonil,
sulfur,
other fungicides, such as acibenzolar-5-methyl, benthiavalicarb, carpropamid, chlorothalonil, cyflufenamid, cymoxanil, diclomezine, diclocymet, diethofencarb, edifenphos, ethaboxam, fenhexamid, fentin acetate, fenoxanil, ferimzone, fluazinam, fosetyl, fosetyl-aluminum, phosphorous acid, iprovalicarb, hexachlorobenzene, metrafenone, pencycuron, penthiopyrad, propamocarb, phthalide, toloclofos-methyl, quintozene or zoxamide,
strobilurins, such as azoxystrobin, dimoxystrobin, enestroburin, fluoxastrobin, kresoxim-methyl, metominostrobin, orysastrobin, picoxystrobin, pyraclostrobin or trifloxystrobin,
sulfenic acid derivatives, such as captafol, captan, dichlofluanid, folpet or tolylfluanid,
cinnamides and analogous compounds, such as dimethomorph, flumetover or flumorph.

### SYNTHESIS EXAMPLES

The procedures described in the synthesis examples below were used to prepare further compounds I by appropriate modification of the starting materials. The compounds thus obtained are listed in the tables below, together with physical data.

### Example 1

### Preparation of 5-methyl-6-(2,4,6-trifluorophenyl)-7-(2-methylpyrrolidin-1-yl)-1,2,4-triazolo[1,5a]pyrimidine

### Example 1a

### 5-Chlorine-6-(2,4,6-trifluorophenyl)-7-(2-methylpyrrolidin-1-yl)-1,2,4-triazolo[1,5a]pyrimidine

A solution of 0.2 g (0.63 mmol) of 5,7-dichloro-6-(2,4,6-trifluorophenyl)-1,2,4-triazolo[1,5a]pyrimidine (cf. WO 98/146607), 0.053 g (0.63 mmol) of 2-methylpyrrolidine and 0.064 g (0.63 mmol) of triethylamine in 5 ml of methylene chloride was stirred at 20-25° C. for about 14 hours. The reaction mixture was then washed with dil. HCl solution and water and then dried and freed from the solvent. 0.2 g of the title compound remained as a colorless crystalline material of m.p. 151-152° C.

¹H-NMR (CDCl₃, 6 in ppm): 8.35 (s, 1H); 6.75-6.9 (m, 2H); 5.15 (m, 1H); 3.2 (m, 1H); 3.05 (m, 1H); 2.3 (m, 1H); 1.75-1.95 (m, 2H); 1.5 (m, 1H); 1.5 (d, 3H)

### Example 1b

### 5-(Dimethylmalon-2-yl)-6-(2,4,6-trifluorophenyl)-7-(2-methylpyrrolidin-1-yl)-1,2,4-triazolo[1,5a]pyrimidine

A solution of 1 g (2.7 mmol) of the 5-chlorotriazolopyrimidine from Example 1a and 1 g (6.5 mmol) of sodium dimethylmalonate in 7 ml of acetonitrile was stirred at 70-80° C. for 4 hours and then at 20-25° C. for about 2.5 days. The resulting precipitate was filtered off and then taken up in methylene chloride and dilute hydrochloric acid. The organic phase was removed and the aqueous phase was extracted two more times with methylene chloride. The combined organic phases were dried and freed from the solvent. 0.6 g of the title compound remained as a yellow oil.

¹H-NMR (CDCl₃, 6 in ppm): 8.4 (s, 1H); 6.9 (t, 1H); 6.8 (t, 1H); 5.1 (m, 1H); 4.65 (s, 1H); 3.8 (s, 6H); 3.15 (m, 1H); 3.0 (m, 1H); 2.25 (m, 1H); 1.7-1.95 (m, 2H); 1.45 (m, 1H); 1.1 (d, 3H)

### Example 1c

### 5-Methyl-6-(2,4,6-trifluorophenyl)-7-(2-methylpyrrolidin-1-yl)-1,2,4-triazolo[1,5a]pyrimidine

0.6 g (1.7 mmol) of the compound from Example 1b in 3 ml of conc. hydrochloric acid was heated under reflux for 3 hours. After cooling to 20-25° C., the reaction mixture was extracted with methylene chloride. The combined organic phases were dried and freed from the solvent. Chromatography on silica gel RP-18 (acetonitrile/water mixtures) gave 0.13 g of the title compound as a colorless solid of m.p. 90 to 93° C.

¹H-NMR (CDCl₃, 8 in ppm): 8.35 (s, 1H); 6.75-6.9 (m, 2H); 5.0 (m, 1H); 3.0-3.2 (m, 2H); 2.3 (s, 3H); 2.25 (m, 1H); 1.75-1.95 (m, 2H); 1.45 (m, 1H); 1.1 (d, 3H)

### Example 2

### Preparation of 5-methyl-6-(2,4,6-trifluorophenyl)-7-(R-1,2-dimethylprop-1-yl)-1,2,4-triazolo[1,5a]pyrimidine [I-7]

### Example 2a

### Methyl 2-(2,4,6-trifluorophenyl)acetate

At −40 to −20° C., 294 ml (0.47 mol) of n-butyllithium solution (1.6 M in hexane) were added to a solution of 76 g (0.47 mol) of hexamethyldisilazane in 440 ml of tetrahydrofuran (THF). At −70° C., 48.85 g (0.25 mol) of 2,4,6-trifluorophenyl acetate, dissolved in 44 ml of THF, were then added dropwise. The mixture was stirred at −70° C. for 2 hours and allowed to warm to 20 to 25° C. over a period of about 14 hours. The reaction mixture was then acidified using 450 ml of ammonium chloride solution and concentrated hydrochloric acid. After dilution with methyl tert-butyl ether (MtBE), the organic phase washed with dilute hydrochloric acid and NaHCO₃ solution and then dried, and the solvent was removed. The residue was fractionated. This gave 34.5 g of the title compound as a light-yellow liquid.

¹H-NMR (CDCl₃, δ in ppm): 13.2 (s, 1H); 6.7 (t, 2H); 3.7 (s, 3H); 2.9 (s, 3H)

### Example 2b

### 5-Methyl-6-(2,4,6-trifluorophenyl)-7-hydroxy-1,2,4-triazolo[1,5a]pyrimidine

For 24 hours, a solution of 34 g (0.134 mol) of methyl 2-(2,4,6-trifluorophenyl)acetate from example 2a and 11.4 g (0.135 mol) of aminotriazole in 150 ml of acetic acid was heated at 120° C. (bath temperature). The acetic acid was then distilled off. The residue crystallized and was digested in diisopropyl ether. This gave 16 g (about 50% pure) of the title compound as a colorless solid which was reacted further without further purification.

¹H-NMR (DMSO-d₆, δ in ppm): 8.35 (s, 1H); 7.35 (t, 2H); 2.2 (s, 3H)

### Example 2c

### 5-Methyl-6-(2,4,6-trifluorophenyl)-7-chloro-1,2,4-triazolo[1,5a]pyrimidine

16 g (about 50% pure) of the triazolopyrimidine from example 2b in 50 ml of phosphorus oxychloride were heated under reflux for 8 hours. The excess phosphorus oxychloride was then distilled off, the residue was dissolved in methylene chloride and the organic phase was stirred with water for about 30 min. The organic phase was then separated off, diluted with a cyclohexane/ethyl acetate mixture and filtered through silica gel, and the solvent was then removed. This gave 6.6 g of the title compound as a colorless solid.

¹H-NMR (CDCl₃, δ in ppm): 8.55 (s, 1H); 6.95 (t, 2H); 2.55 (s, 3H)

### Example 2d

### 5-Methyl-6-(2,4,6-trifluorophenyl)-7-(R-1,2-dimethylprop-1-yl)-1,2,4-triazolo[1,5a]pyrimidine

A solution of 0.5 g (1.7 mmol) of the 7-chlorotriazolopyrimidine from example 2c, 0.2 g (2.5 mmol) of R-1,2-dimethylpropylamine and 0.3 g (3 mmol) of triethylamine in 5 ml of methylene chloride was stirred at 20 to 25° C. for about 14 hours. The reaction mixture was then washed with water, and the solvent was removed. The residue was purified by preparative MPLC using acetonitrile/water 60:40 on silica gel RP 18. The eluate gave, after removal of the solvent by distillation, 0.4 g of the title compound as a colorless crystalline material of m.p. 113-115° C.

¹H-NMR (CDCl₃, δ in ppm): 8.3 (s, 1H); 6.85 (t, 2H); 6.1 (d, broad, 1H); 3.1 (m, 1H); 2.3 (s, 3H); 1.6 (m, 1H); 1.05 (d, 3H); 0.8 (d, 6H)

TABLE I
Compounds of the formula I

| | | | | | | Phys. Data |
|---|---|---|---|---|---|---|
| No. | R¹ | R² | L¹ | L² | X | (m.p. [° C.]; ¹H-NMR [ppm]) |
| I-1 | —CH(CH₃)—CH═CH—CH(CH₃)— | | F | F | CH₃ | 8.45(s); 6.85(t); 5.75(s); |
| | | | | | | 4.85(q); 2.4(s); 1.1(d) |
| I-2 | —CH(CH₃)—(CH₂)₃— | | F | F | CH₃ | 90-93 |
| I-3 | —CH(CH₃)—(CH₂)₃— | | Cl | H | CH₃ | 8.3(s); 7.3-7.5(m); 7.15(m); |
| | | | | | | 5.25(m); 4.85(m); 1.15(d); |
| | | | | | | 1.05(d) |
| I-4 | —CH(CH₃)—(CH₂)₃— | | F | H | CH₃ | 8.35(s); 7.5(m); 7.1(t); 7.0(t); |
| | | | | | | 5.0(m); 1.1(d) |
| I-5 | —CH₂—CH(CH₃)—(CH₂)₃— | | F | H | CH₃ | 8.35(s); 7.45(m); 7.05(t); |
| | | | | | | 3.55(m); 0.75(d) |
| I-6 | —CH(CH₃)—CH₂CH₃ | H | F | F | CH₃ | 104-106 |
| I-7 | R—CH(CH₃)—CH(CH₃)₂ | H | F | F | CH₃ | 113-115 |
| I-8 | —(CH₂)₂—CF═CF₂ | H | F | F | CH₃ | 139-142 |

Owing to the hindered rotation of the 6-phenyl group, in the case of unsymmetric phenyl substitution, two diastereomers may be present, which may differ in their physical data.

Examples of the action against harmful fungi

The fungicidal action of the compounds of the formula I was demonstrated by the following experiments:

The active compounds were prepared as a stock solution with 25 mg of active compound which was made up to 10 ml with a mixture of acetone and/or DMSO and the emulsifier Uniperol® EL (wetting agent having emulsifying and dispersing action based on ethoxylated alkylphenols) in a volume ratio solvent/emulsifier of 99 to 1. The solution was then made up to 100 ml with water. This stock solution was diluted to the active compound concentration stated below using the solvent/emulsifier/water mixture described.

Use Example 1—Activity Against Gray Mold on Bell Pepper Leaves caused by Botrytis cinerea, Protective Application

Bell pepper seedlings of the cultivar “Neusiedler Ideal Elite” were, after 2 to 3 leaves were well developed, sprayed to run off point with an aqueous suspension having the concentration of active compounds stated below. The next day, the treated plants were inoculated with a spore suspension of Botrytis cinerea which contained 1.7×10⁶ spores/ml in a 2% strength aqueous biomalt solution. The test plants were then placed in a dark climatized chamber at 22 to 24° C. and high atmospheric humidity. After 5 days, the extent of the fungal infection on the leaves could be determined visually in %.

In this test, the plants which had been treated with 250 ppm of the compounds I-1, I-2, I-3, I-4, I-5, I-6, I-7 or I-8 showed an infection of at most 1%, whereas the untreated plants were 90% infected.

Use Example 2—Activity Against Mildew on Cucumber Leaves caused by Sphaerotheca fuliginea, 3 day Protective Application

At the cotyledon stage, leaves of potted cucumber seedlings were sprayed to run off point with an aqueous suspension having the concentration of active compounds stated below. 3 days after the application, the plants were inoculated with an aqueous spore suspension of mildew of cumber (Sphaerotheca fuliginea). The plants were then cultivated in a greenhouse at temperatures between 20 and 24° C. and at 60 to 80% relative atmospheric humidity for 7 days. The extent of the mildew development was then determined visually in % infection of the cotyledon area.

In this test, the plants which had been treated with 250 ppm of the compounds I-3 or I-4 showed no infection, whereas the untreated plants were 100% infected.

1. A 6-phenyltriazolopyrimidine of the formula I 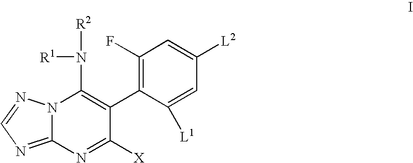
in which the substituents are as defined below:
R1 is C4-C8-alkyl, C4-C8-haloalkyl, C3-C6-cycloalkyl substituted by at least one group Ra, C3-C8-halocycloalkyl, C3-C6-cycloalkyl-C1-C4-alkyl, C5-C8-alkenyl, C2-C8-haloalkenyl, C3-C6-cycloalkenyl, C3-C6-halocycloalkenyl, C2-C8-alkynyl, C2-C8-haloalkynyl or phenyl, naphthyl, or a five- or six-membered saturated, partially unsaturated or aromatic heterocycle which contains one to four heteroatoms from the group consisting of O, N and S,
R2 is hydrogen, C1-C3-alkyl or one of the groups mentioned under R1,
R1 and R2 together with the nitrogen atom to which they are attached may also form a five- to eight-membered saturated or partially unsaturated heterocyclyl or a five- or six-membered heteroaryl which is attached via N and may contain one to three further heteroatoms from the group consisting of O, N and S as ring member and/or may carry one or more substituents from the group consisting of halogen, C1-C6-alkyl, C1-C6-haloalkyl, C2-C6-alkenyl, C2-C6-haloalkenyl, C1-C6-alkoxy, C1-C6-haloalkoxy, C3-C6-alkenyloxy, C3-C6-haloalkenyloxy, (exo)-C1-C6-alkylene and oxy-C1-C3-alkyleneoxy,
except piperidin-1-yl, which is unsubstituted or substituted by one or more methyl groups;
R1 and/or R2 may carry one to four identical or different groups Ra:
Ra is halogen, cyano, nitro, hydroxyl, C1-C6-alkyl, C1-C6-haloalkyl, C1-C6-alkylcarbonyl, C3-C6-cycloalkyl, C1-C6-alkoxy, C1-C6-haloalkoxy, C1-C6-alkoxycarbonyl, C1-C6-alkylthio, C1-C6-alkylamino, di-C1-C6-alkylamino, C2-C8-alkenyl, C2-C8-haloalkenyl, C2-C6-alkenyloxy, C2-C8-alkynyl, C2-C8-haloalkynyl, C3-C6-alkynyloxy, oxy-C1-C3-alkyleneoxy, C3-C8-cycloalkenyl, phenyl, naphthyl, a five- or six-membered saturated, partially unsaturated or aromatic heterocycle which contains one to four heteroatoms from the group consisting of O, N and S, where these aliphatic, alicyclic or aromatic groups for their part may be partially or fully halogenated;
L¹ is chlorine or fluorine;
L² is hydrogen,
is, if L¹ is fluorine, also fluorine;
X is C₁-C₄-alkyl

2. The compound of the formula I according to claim 1, in which L1 and L2 are fluorine.

3. The compound of the formula I according to claim 1, in which L1 is fluorine and L2 is hydrogen.

4. The compound of the formula I according to claim 1, in which L1 is chlorine.

5. The compound of the formula I according claim 1, in which R1 and R2 together form a pyrrolidine ring which may carry one to four identical or different groups Ra.

6. A compound of the formula I.1: 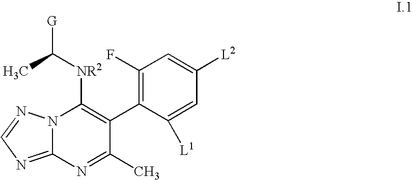
in which
G is C2-C6-alkyl, C1-C4-alkoxymethyl or C3-C6-cycloalkyl;
R2 is hydrogen or methyl; and
L1 and L2 are as defined in claim 1.

7. A compound of the formula I.2, 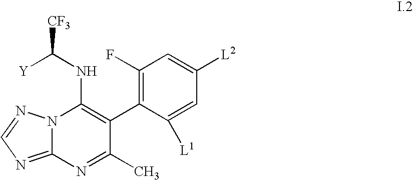
in which Y is C2-C6-alkyl and L1 and L2 are as defined in any of claim 1.

8. A compound of the formula I.3, 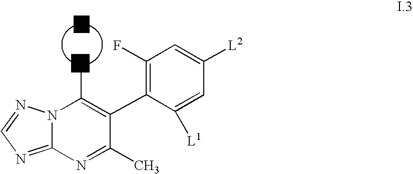
in which
D together with the nitrogen atom forms a five- or six-membered saturated or partially unsaturated heterocyclyl or heteroaryl which is attached via N and may contain a further heteroatom from the group consisting of O, N and S as ring member and/or may carry one or more substituents from the group consisting of halogen, C1-C6-alkyl, C1-C6-haloalkyl, C2-C6-alkenyl, C2-C6-haloalkenyl, C1-C6-alkoxy, C1-C6-haloalkoxy, C3-C6-alkenyloxy, C3-C6-haloalkenyloxy, (exo)-C1-C6-alkylene and oxy-C1-C3-alkyleneoxy;
except piperidin-1-yl, which is unsubstituted or substituted by one or more methyl groups;
L¹ and L² are as defined in claim 1.

9. The compound of the formula I according to claim 1, in which the variables are as defined below:
L1,L2 are fluorine, L3 is hydrogen; X is methyl; and
L1,L2 are chlorine, L3 is hydrogen; X is methyl.

10. A process for preparing the compound of the formula I according to claim 1, by reacting 5-amino1,2,4-triazole of the formula II 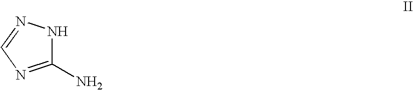
with a keto ester of the formula III 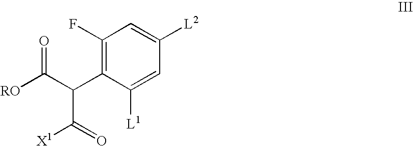
in which R is C1-C4-alkyl to give a 7 hydroxytriazolopyrimidine of the formula IV, 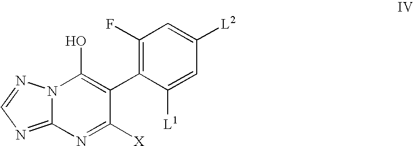
which is, using a halogenating agent, converted into the corresponding 7 halotriazolopyrimidine of the formula V 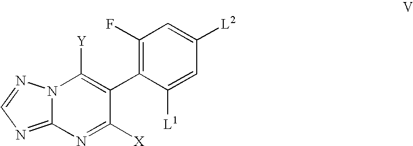
and compound V is reacted with an amine of the formula VI 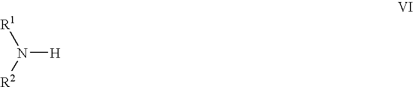
to give the compound of the formula I.

11. A compound of the formulae IV and V:
5-methyl-6-(2-chloro-6-fluorophenyl)-[1,2,4]triazolo[1,5-a]pyrimidin-7-ol;
7-chloro-5-methyl-6-(2-chloro-6-fluorophenyl)-[1,2,4]triazolo[1,5-a]pyrimidine;
7-bromo-5-methyl-6-(2-chloro-6-fluorophenyl)-[1,2,4]triazolo[1,5-a]pyrimidine;
5-methyl-6-(2,6-difluorophenyl)-[1,2,4]triazolo[1,5-a]pyrimidin-7-ol;
7-chloro-5-methyl-6-(2,6-difluorophenyl)-[1,2,4]triazolo[1,5-a]pyrimidine;
7-bromo-5-methyl-6-(2,6-difluorophenyl)-[1,2,4]triazolo[1,5-a]pyrimidine;
5-methyl-6-(2,4,6-trifluorophenyl)-[1,2,4]triazolo[1,5-a]pyrimidin-7-ol;
7-chloro-5-methyl-6-(2,4,6-trifluorophenyl)-[1,2,4]triazolo[1,5-a]pyrimidine;
7-bromo-5-methyl-6-(2,4,6-trifluorophenyl)-[1,2,4]triazolo[1,5-a]pyrimidine.

12. A process for preparing a compound of the formula I according to claim 1 by reacting a 5-halotriazolopyrimidine of the formula VII 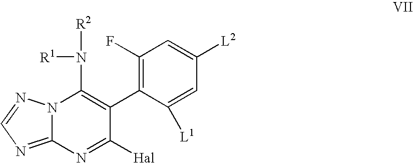
with a malonate of the formula VIII, 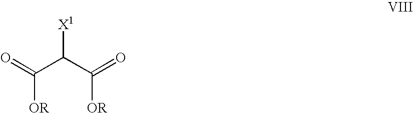
in which X1 is a hydrogen or C1-C4-alkyl, to give a compound of the formula IX 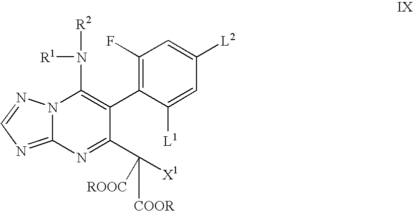
which, after decarboxylation, gives the compound of the formula I.

13. A composition, comprising a solid or liquid carrier and a compound of the formula I according to claim 1.

14. Seed, comprising a compound of the formula I according to claim 1 in an amount of from 1 to 1000 g/100 kg.

15. A method for controlling phytopathogenic harmful fungi, which method comprises treating the fungi or the materials, plants, the soil or seed to be protected against fungal attack with an effective amount of a compound of the formula I according to claim 1.
